(19) United States
(12) Patent Application Publication
(10) Pub. No.: US 2013/0218104 A1
(43) Pub. Date: Aug. 22, 2013

(54) DEVICES AND METHODS FOR DELIVERY OF AGENTS TO BIOLOGICAL TISSUE

(71) Applicant: Stephen J. Smith, Cooperstown, NY (US)

(72) Inventor: Stephen J. Smith, Cooperstown, NY (US)

(21) Appl. No.: 13/752,313

(22) Filed: Jan. 28, 2013

Related U.S. Application Data
(60) Provisional application No. 61/600,518, filed on Feb. 17, 2012.

(51) Int. Cl. A61M 35/00

(52) U.S. Cl. CPC A61M 35/00
USPC 604/290; 604/310

(57) ABSTRACT

Described herein are systems, apparatuses, and methods that employ a medical device to augment delivery of an agent to biological tissue. The medical device includes a reservoir containing the agent, a release port, and sealable base that can be attached to the biological tissue so that the agent is held under pressure upon attachment. The device can only release the agent through the release port. A penetrating instrument can be utilized to augment delivery of the agent to the biological tissue.

### TECHNICAL FIELD

This disclosure relates generally to the delivery of pharmaceutical and/or diagnostic agents to biological tissue.

### BACKGROUND

The development of pharmaceutical and diagnostic agents has progressed at a rapid pace over the last decade. With this development, the question of how to maximize therapeutic benefit while minimizing systemic toxicity has been increasingly emphasized. Delivering agents directly to affected tissues and organs provides the most therapeutic benefit with the least systemic damage; however, focal delivery remains a challenge for a number of reasons due at least in part to risks inherent in the delivery of these agents.

The description of deficiencies of conventional delivery of pharmaceutical and diagnostic agents is merely intended to provide an overview of some of the problems of current agent delivery methods, and is not intended to be exhaustive. Other problems with the state of the art, and the corresponding benefits of some of the various non-limiting embodiments described herein, may become further apparent upon review of the following detailed description.

### SUMMARY

The following presents a simplified summary to provide a basic understanding of some aspects described herein. This summary is not an extensive overview of the disclosed subject matter. It is not intended to identify key or critical elements of the disclosed subject matter, or delineate the scope of the subject disclosure. Its sole purpose is to present some concepts of the disclosed subject matter in a simplified form as a prelude to the more detailed description presented later.

Described herein are systems, apparatuses, and methods that facilitate delivery of an agent to a biological tissue. According to an embodiment, a device is described that can include a reservoir and a sealable base. The reservoir can hold the agent and can be impermeable to release of the agent except at a release port. The structure of the reservoir can allow more than one agent to be contained in the reservoir at one time. The release port can facilitate delivery of the agent to the biological tissue. The structure of the release port can vary based on the material property of the agent. The device can also include a sealable base connected to the reservoir, such that when the device is sealed to the biological tissue, the agent in the reservoir can be held under pressure. The sealable base can include an attachment mechanism that can facilitate an attachment to the biological tissue for a short or an extended period of time. In another embodiment, the device can employ an injection port to facilitate delivery of the agent to the biological tissue via a temporary channel created by penetration and subsequent removal of a penetrating device.

In a further embodiment, a method for delivering an agent to biological tissue is described. The method includes attaching a device to a biological tissue. After the device is attached to the biological tissue, the device and the biological tissue can be penetrated with a penetrating instrument. The penetrating instrument can be removed from the biological tissue and the device, thereby augmenting delivery of the agent to the biological tissue.

The following description and the annexed drawings set forth in detail certain illustrative aspects of the disclosed subject matter. These aspects are indicative, however, of but a few of the various ways in which the principles of the innovation may be employed. The disclosed subject matter is intended to include all such aspects and their equivalents. Other advantages and distinctive features of the disclosed subject matter will become apparent from the following detailed description of the innovation when considered in conjunction with the drawings.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 is a schematic top view illustration of several examples of a medical device that can facilitate delivery of an agent to biological tissue.

FIG. 2 is a schematic cross-sectional illustration of a medical device attached to a biological tissue to facilitate delivery of an agent to the biological tissue.

FIG. 3 is a schematic cross-sectional illustration of an example of a medical device that can facilitate delivery of an agent to biological tissue via a release port.

FIG. 4 is a schematic cross-sectional illustration of an example of a medical device that can facilitate delivery of an agent to biological tissue via an injection.

FIG. 5 is a schematic cross-sectional illustration of a medical device that can facilitate delivery of an agent to biological tissue via a channel formed at the injection site.

FIG. 6 is a schematic illustration of an example use of the medical device.

FIG. 7 is a schematic process flow diagram of a method for delivering an agent to biological tissue.

FIG. 8 is a schematic process flow diagram of a method for augmenting delivery of an agent to biological tissue.

FIG. 9 is a schematic process flow diagram of an example method for delivery of an agent to an eye.

### DETAILED DESCRIPTION

In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Described herein are devices and methods that can facilitate the delivery of pharmaceutical and/or diagnostic agents to biological tissue. The devices and methods can increase safety and/or augment delivery of an agent to biological tissue when the agent is delivered via penetration through the device holding the agent under pressure and at least partially through the biological tissue. The devices and methods provide localized treatments and/or diagnostic procedures with improved safety profiles and/or potentiated results compared to traditional treatments and/or diagnostic procedures alone. The devices and methods can improve the safety profiles and/or potentiate the results through treating a compromised tissue barrier that results following certain therapeutic and/or diagnostic procedures, thereby minimizing potential complications and side effects.

Reference throughout this specification to “various embodiments,” “one embodiment,” or “an embodiment,” means that a particular feature, structure, or characteristic described in connection with the embodiment is included in at least one embodiment. Thus, the appearances of the phrase “in one embodiment,” or “in an embodiment,” in various places throughout this specification are not necessarily all referring to the same embodiment. Furthermore, the particular features, structures, or characteristics may be combined in any suitable manner in one or more embodiments.

The words “exemplary” and “example” are used herein to mean serving as an example, instance, or illustration. For the avoidance of doubt, the subject matter described herein is not limited by such examples. In addition, any aspect or design described herein as “exemplary” is not necessarily to be construed as preferred or advantageous over other aspects or designs, nor is it meant to preclude equivalent structures and techniques known to those of ordinary skill in the art. Furthermore, to the extent that the terms “includes,” “has,” “contains,” and other similar words are used in either the detailed description or the claims, such terms are intended to be inclusive—in a manner similar to the term “comprising” as an open transition word—without precluding any additional or other elements.

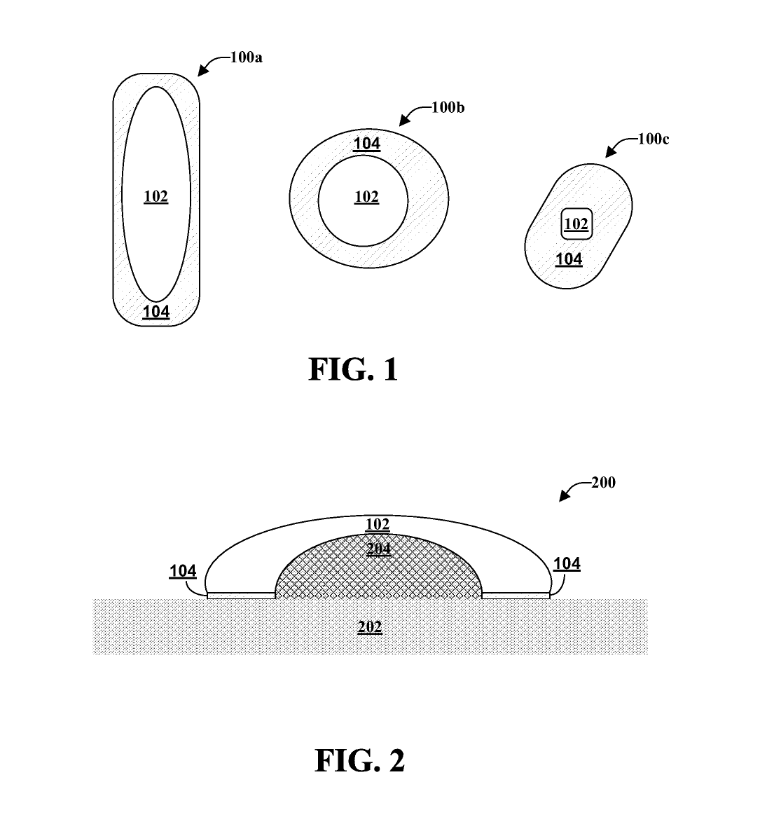

Referring now to FIG. 1, illustrated is medical device that can be constructed in various shapes 100a, 100b, 100c. The medical device can be constructed in any shape and/or size sufficient to facilitate the delivery of an agent to biological tissue. The size and shape of the medical device can depend, for example, on the size of the biological tissue, the amount of agent necessary for the biological tissue, the size of the penetrating device penetrating through the medical device, and the like. It will be understood that examples 100a, 100b, and 100c are intended to show that the size and shape of the medical device can vary; these examples are not intended to limit the size and shape of the medical device in any way. For example, the device can have a shape of a simple tube, or a shape of a complex form that covers a large surface area, such as a broad, wide device with one or more phalanges.

The medical device can be implanted within the biological tissue or attached to the surface of the biological tissue. According to an embodiment, the medical device (or any portion of the medical device) can be biodegradable, formed, for example, of biodegradable materials, such as polymers. The biodegradable material can dissolve, disintegrate, or the like upon contacting biological tissue. At least a part of the medical device can be shaped to fit against and/or mirror an anatomical structure of the biological tissue.

The medical device includes a reservoir 102 and a sealable base 104. The reservoir 102 can be any type of reservoir that can hold an agent. The reservoir 102 can be of any size or shape sufficient to hold a predetermined amount of an agent. The reservoir 102 can include a low-profile housing with a minimal height dimension and a minimal lateral dimension constrained only by the ability to hold a predetermined amount of agent and/or a necessary pressure within the reservoir. For example, the reservoir 102 can have a generally spherical shape so that the medical device resembles approximately half of a sphere.

When used herein, the term agent can refer to any chemical that has a biological application. For example, an agent can be a diagnostic agent, a therapeutic agent, or any combination thereof. A therapeutic agent can be any drug, compound, composition, or the like, recognized by the United States Pharmacopeia, the National Formulary, or any other pharmacopeia publication in any country; therapeutic agents can also include any agent that is under development either currently or in the future. Therapeutic agents can also include any supplements currently on the market, including homeopathic supplements. Therapeutic agents also include any agent that can be used to reduce the risk of infection, including any agent that facilitates sterilization, such as betadine, povidone-iodine, or any similar agent.

The agent can be a liquid, a solid, a gel, a viscous material, a solution, a suspension, or the like. Within the medical device, communication between the agent and a biological tissue of interest only occurs when the agent is released from the reservoir 102 (e.g., through a release port). The medical device can facilitate prophylactic use of the agent to improve or increase a safety profile of the agent administered through different devices, procedures, or the like. For example, a therapeutic agent administered through the medical device described herein can have an improved safety profile compared to a therapeutic agent administered via injection not given through an otherwise sealed and pressurized device.

The term “reservoir” can refer to any number of reservoirs that can house any number of agents. Although the medical device FIG. 1 is shown to include just one reservoir 102, this is simply for ease of illustration. The medical device can include any number of reservoirs 102. For example, the medical device can include a first reservoir that can include a first agent and a second reservoir that can include a second agent.

The reservoir 102 can be made of any material that is approved by the U.S. Food and Drug Administration for contact with biological tissue. In an embodiment, the reservoir 102 can be made of a material impermeable to the agent so as to preclude release of the agent. According to another embodiment, the reservoir 102 can include a portion that is impermeable to the agent and a portion that is permeable to the agent to facilitate administration of the agent to biological tissue. At least part of the reservoir 102 can be made of a material with elastomeric characteristics so that the reservoir 102 can increase or decrease in size depending on an amount of agent in the reservoir 102, a pressure change within the reservoir 102, or the like. The reservoir 102 can also be made of a material that exhibits no elastomeric properties, such as a rigid material, a semi-rigid material, or the like.

The reservoir 102 can hold the agent prior to attachment to the biological tissue. The reservoir 102 can also be filled with the agent after attachment to the biological tissue. The reservoir 102 can include markings, indications, or the like that can facilitate administration of the agent to the reservoir or to the biological tissue. For example, the reservoir 102 can include markings that indicate an amount of agent held in the reservoir.

The medical device also includes a sealable base 104 that is connected to the reservoir 102. The sealable base 104 can be of any shape or size to facilitate attachment of the medical device to the biological tissue. The sealable base 104 can cover any amount of the reservoir 102 sufficient to facilitate attachment of the medical device to the biological tissue. For example, the sealable base 104 can extend internally with an internal diameter smaller than the widest diameter of the reservoir 102. The sealable device can be an air, fluid, or gel filled tube, a disk, a disc with an internal groove, a shape that facilitates a suction type of seal, or any other shape that can facilitate attachment to the biological tissue.

The sealable base 104 can employ an attachment mechanism that can facilitate attachment of the medical device to the biological tissue. The attachment mechanism can be of any number of different shapes, contours, sizes, and the like to allow for a tight seal against any different biological tissue at varying pressures. For example, the sealable base 104 can be curved downward, inward, or the like all the way around so that when the medical device is positioned on the biological tissue and pressure is applied to the device, the sealable base can flatten and expand outward or inward slightly with a corresponding increase in pressure exerted by the sealable base 104 against the tissue, enabling a tight seal.

The attachment mechanism can be any mechanism that enables stable contact with the biological tissue. This stable contact can occur for an extended, prolonged, or the like time period (e.g., days, weeks, months or years), allowing multiple injections through it before it must be replaced or removed. The attachment mechanism can also be any mechanism that enables the medical device to be attached and removed from the biological tissue. The attachment and removal can be rapid. Rapid attachment and removal refers to any method of attachment and removal that does not require an extensive procedure. For example, a rapid method of attachment and removal may take seconds or minutes where other methods of attachment and removal may take several hours.

The attachment mechanism can be any mechanism that can facilitate a watertight seal between the medical device and the biological tissue. When used herein, the term “watertight” refers to any seal that is impervious to liquid or gelatinous material. The attachment mechanism can include an adhesive material. The attachment mechanism can also utilize manual pressure as the primary or secondary means of attachment and sealing. The attachment mechanism can also utilize a suture with one end of the suture anchored in the biological tissue and the other end of the suture anchored to a suture holder attached to the medical device (e.g., attached to a housing of the medical device), enabling surgical fixation of the medical device to the biological tissue.

Referring now to FIG. 2, illustrated is a cross-sectional view of a medical device 200 sealed to a biological tissue 202. The medical device 200 can provide a pressurizable system, sealed to the biological tissue 202, that can be used to facilitate delivery of any number of agents or materials 204 through the medical device 200 to the biological tissue 202 (e.g., through injection or similar action). The shape of the medical device 200 can be dictated by a shape or size of the biological tissue 202, an amount of the agent 204, or the like.

The medical device 200 includes a reservoir 102 that can hold an agent 204. The reservoir 102 can be made of a rigid, semi-rigid, elastomeric, or other material to provide structural support for the device, to hold the agent, or the like. The medical device 200 also includes a sealable base 104 that can be shaped in a way to facilitate ease of attachment of the medical device 200 to the biological tissue 202. For example, if the biological tissue 202 is the eye, the sealable base 104 can be formed in a way that complements the curvature of the external surface of the eye. The sealable base 104 can be any size relative to the bottom of the reservoir 102. The sealable base 104 can be wider than the bottom of the reservoir 102, approximately the same size as the bottom of the reservoir 102, smaller than the bottom of the reservoir 102, or any size relative to the bottom of the reservoir 102 that facilitates attaching the medical device 200 to the biological tissue 202.

The sealable base 104 can be made of one or more materials that are rigid, semi-rigid, elastomeric, or any combination thereof. The sealable base 104 can be made of a material that is the same or different from the material used to construct the reservoir 102. The sealable base 104 can have some flexibility that can allow for sealing against an irregularly shaped biological tissue 202.

The underside of the sealable base 104 comes into contact with the biological tissue 202. The underside of the sealable base 104 can employ an attachment mechanism to attach to the biological tissue 202. The attachment mechanism can employ a bioadhesive material including any adhesive material that is or will be approved by the U.S. Food and Drug Administration for biological contact. For example, the bioadhesive material can include a cyanoacrylate derivative to promote a tight seal against the biological tissue 202. The sealable base 104 can utilize a mechanical mechanism, a suction mechanism, a suture mechanism, or any other mechanism that can facilitate attachment to facilitate a tight seal to the biological tissue 202. Any attachment mechanism employed by the sealable base can allow for a tight seal, even with pressure changes that occur from filling or emptying the reservoir 102.

The sealable base 104 can include an elastomeric material. The elastomeric material can accommodate distension and/or compression of the medical device 200, depending on the amount of agent 204 present in the medical device 200. The sealable base 104 and the reservoir 102 can be connected in a fluid tight manner.

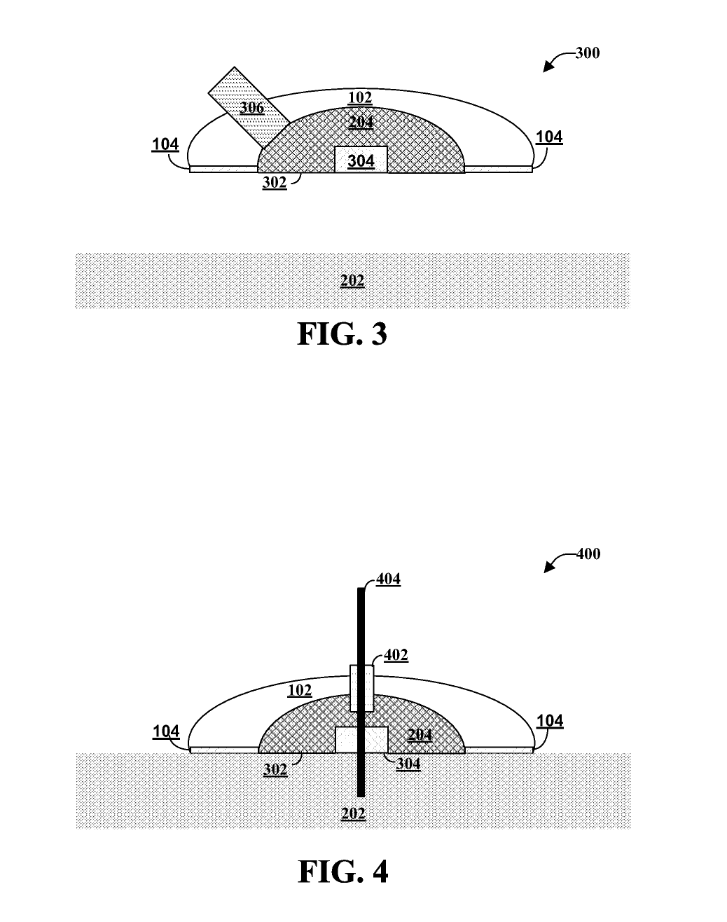

Referring now to FIG. 3, illustrated is a medical device 300 that is not yet sealed to a biological tissue 202. The medical device can facilitate delivery of an agent 204 to the biological tissue 202 upon attachment to the biological tissue 202. The medical device includes a reservoir 102 and a sealable base 104. The reservoir 102 can hold the agent 204 and be impenetrable to the agent 204 along the whole surface of the reservoir 302, except at a release port 304.

The release port 304 can be any outlet for the agent 204. The release port can be bordered by the attachment mechanism of the sealable base 104 or the impermeable portion of the reservoir 302 so that the release port 304 can be attached to the biological tissue 202. The release port 304 can be open to the biological tissue at some point during the use of the medical device 300 to facilitate delivery of the agent 204 to the biological tissue 202. The release port can have any shape, limited only by properties of the agent 204 (e.g., molecule size, diffusion properties, and the like), or by properties of a penetrating device (e.g., size, shape, and the like). For example, the size of the release port 304 can be varied to control an area exerting pressure on the biological tissue 202. The diffusion rate, infusion rate and the like through the release port 304 can be influenced by the size of the release port 304. The release port can, for example, be substantially smaller than the reservoir 102, which can allow the reservoir walls 102 to function as a funnel that opens to a small release port 304.

The release port 304 can be open before the medical device 300 is attached to the biological tissue 202. The medical device 300 can be filled with the agent 204 following attachment to the biological tissue 202. The medical device 300 can also be filled, refilled or emptied while attached to the biological tissue. The pressure in the medical device 300 can be modified by varying the amount of agent 204 included in the reservoir.

The release port 304 can be closed until the medical device 300 is attached to the biological tissue. The release port 304 can include a structural element that retains the agent in the reservoir prior to the attachment to the biological tissue 202. For example, the release port 302 can be covered with an impermeable or semi-permeable covering that can eventually allow communication between the agent 204 and the biological tissue 202 abutting the release port 302. The covering can allow the medical device 300 to carry the agent 204 at a predetermined pressure for single or multiple uses. The release port 304 can open to the biological tissue 202 after the medical device 300 is attached to the biological tissue 302 to facilitate delivery of the agent 204 to the biological tissue 202 (e.g., by diffusion). For example, the release port 304 can be covered with a biodegradable material that can degrade following contact with the biological tissue 202, or the release port 304 cover can be manually opened following the penetration of the medical device 300 and release port 304 with a penetrating instrument. When the release port 304 is closed prior to attachment, the medical device 300 can be manufactured in a prepackaged dosage format with the appropriate amount of the agent 204 already in the reservoir.

The reservoir 102 can also include a refill port 306 that can facilitate addition or removal of the agent 204 to or from the reservoir 102. The refill port 306 can enable the reservoir 102 to be filled, refilled, emptied, or the like. The filling, refilling, emptying, or the like can occur when the medical device 300 is not attached to the biological tissue 202. The filling, refilling, emptying, or the like can occur when the medical device 300 remains attached to the biological tissue 202. The refill port 306 can also serve as an injection port. The refill port can be covered by a material that allows penetration with a penetrating device (e.g., a needle or similar instrument), while self-sealing upon removal of the penetrating instrument, thereby preventing leakage of the agent 204 from the reservoir after the penetrating device has been completely withdrawn from the device.

Although a single release port 304 and a single refill port 306 are illustrated, this is only for simplicity of illustration. It will be understood that the medical device 300 can include any number of release ports, refill ports and/or injection ports. The release ports can be located at any point on the reservoir surface 302. The refill ports and/or injection ports can be located at any point on the reservoir 102.

Referring now to FIG. 4, illustrated is a cross-sectional view of a medical device 400 attached to a biological tissue 202. The medical device 400 can augment delivery of an agent 204 to the biological tissue 202. The medical device includes a reservoir 102 and a sealable base 104. The reservoir 102 can hold the agent 204 and be impenetrable to the agent 204 along the whole surface of the reservoir 302, except at a release port 304.

The medical device 400 can include an injection port 402. Although the injection port 402 is illustrated at a center position of the reservoir, it will be understood that the injection port 402 can be located in any position along the reservoir. For example, the injection port 402 can be located off center to enable greater ease in giving beveled injections. The injection port 402 can also be located centrally over the central portion of the reservoir 102. The injection port 402 can be positioned on the reservoir 102 to cover a potential space, hole, or the like in the reservoir 102.

The injection port 402 can be attached to the reservoir 102 in such a way that it forms a fluid tight seal with the reservoir 102. The injection port 402 can be covered with a material that prevents the agent 204 from escaping from the reservoir 102, but facilitates penetration by a penetrating instrument 404. The cover injection port 402 can include a self-sealing material that can self-seal upon removal of the penetrating instrument 404, so that the agent 204 cannot escape from the reservoir 102. The self-sealing material can be silicone elastomer, latex, synthetic rubber, or the like. A dome or concavity can exist over the central portion of the injection port, which can be utilized to improve ease of injections or similar procedures.

The injection port 402 can have functionality similar to the refill port 306. The injection port 402 can allow the agent 202 in the reservoir 102 to be filled, refilled, or emptied. The injection port 402 can allow penetration by a penetrating device 404 to augment traditional diagnostic and/or treatment modalities.

The injection port 402 can facilitate penetration of the device 400 and the biological tissue 202 with a penetrating instrument 404. Penetration with the penetrating device can facilitate delivery of the agent 204 to the biological tissue 202. The penetration can allow the agent 204 to be used prophylactically to increase or improve the safety profile of procedures that penetrate through the medical device and the biological tissue or to potentiate the effectiveness of the procedures.

The shape of the medical device 400 can be varied to allow attachment to any penetrating instrument to create an injection system. For example, the medical device 400 can be integrally attached to a needle or ozurdex injection system. This injection system can be attached to biological tissue via the sealable base of medical device 400 with subsequent penetration through the medical device 400 and biological tissue by the penetrating instrument.

The shape of the medical device 400 can be varied so to allow penetration through the device by penetrating instruments 404 of different shapes and sizes. For example, the penetrating instrument 404 can be a high or low gauge needle, an ozurdex injection system, an implantable drug delivery system, a virectomy instrument, a microsurgical tool, injections of intraocular free floating delivery devices, scleral fixated intravitreal implants, and intravitreal injections of solutions, suspensions, or verisome™ or the like. The injection port 402 can have markings that facilitate administration of the agent 204 to the biological tissue 202.

The device 400 can, for example, be a pre-sealed, pre-filled device with an injection port made of translucent material. Depending on the agent 204 within the reservoir, the translucent injection port 402 can allow for direct visualization of the biological tissue 202 located under the release port 304, which can enable greater precision in giving an injection or other procedure. The injection port 402, whether translucent or opaque, can also contain markings, indentations or other means that help in delineating where the penetrating device penetrates the biological tissue. For example, the device can mark millimeters from a biological landmark, such as the limbus of the eye, to aid in giving intravitreal injections.

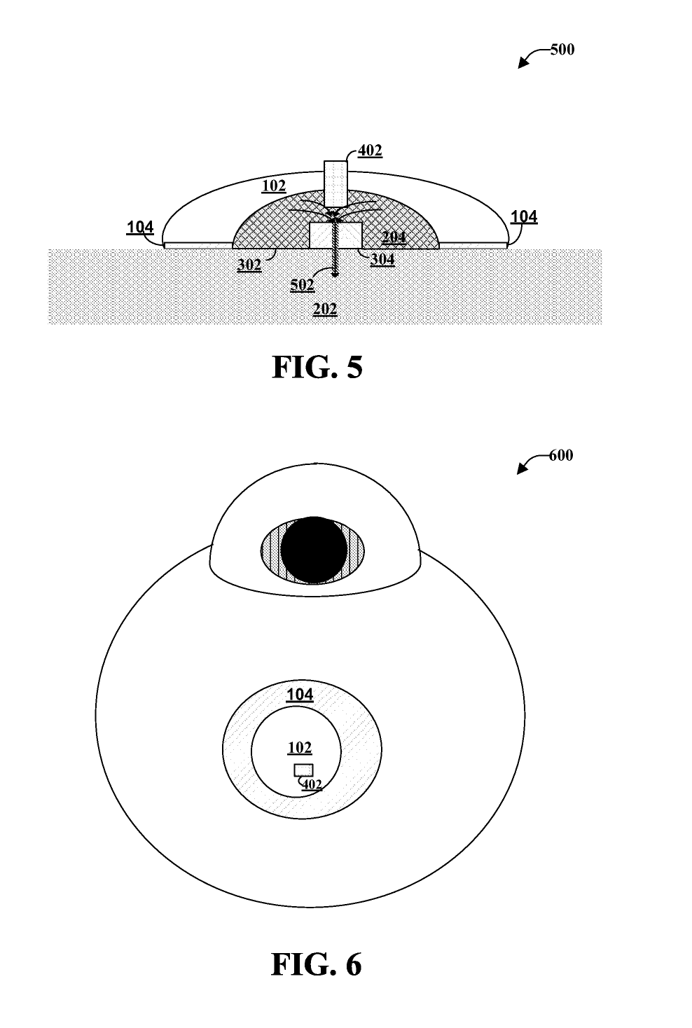

Referring now to FIG. 5, illustrated is a medical device 500 attached to a biological tissue 202 after removal of a penetrating instrument. The penetrating instrument penetrated through the medical device 500 and a portion of the biological tissue 202, leaving a channel 502 through the release port 304 and the biological tissue 202. The channel 502 can be a temporary channel because the biological tissue 202 can self repair.

The medical device 500 can employ a pressurized system to facilitate delivery of the agent 204 to the biological tissue 202 through the temporary channel 502 (as shown by the arrows in FIG. 5). The temporary channel 502 can be created by penetration and subsequent removal of the penetrating device. The temporary channel 502 can be bathed in the agent 204 from the reservoir 102. The temporary channel 502 can be a source of toxicity to the biological tissue 202, including infection, tumor spread, and more. Depending on the pressure of the agent 204 inside the device 500, the agent 204 will be forced into the channel 502 following removal of the penetrating instrument 404, treating the biological tissue 202. The pressure inside the device 500 will vary based on the characteristics of the channel, the amount of agent contained in the medical device 500, and the pressure exerted by the biological tissue 202 against the agent 204 under pressure. For example, following medical device 500 attachment to the eye, penetration and removal of a device through the device and ocular tissue (into the vitreous), the pressure inside the device (arrows in FIG. 5) will eventually equilibrate with the pressure inside the eye. Based on the pressure inside the device 500 and the pressure inside the eye, a certain amount of the agent can be forced through the channel 502 prior to pressure equilibration. In addition, pressure changes can occur in the device 500 while the device 500 is attached to the biological tissue 202 due to diffusion through the release port. These pressure changes will be augmented following the penetrating procedure.

Parts of the reservoir 102, the injection port 402, and/or the release port 304 can be constructed from an elastic material that allows penetration by the penetrating device, while preventing the agent 204 from dissipating following removal of the penetrating device. The size and pressure inside the device 500 can be modified by varying the amount of agent contained in the device or by exerting manual pressure on the device to allow for alterations of the amount of agent 204 inside the device 500 that would be forced into the temporary channel 502 and biological tissue 202 following an injection or similar procedure.

Referring now to FIG. 6, illustrated is an example 600 of a medical device attached to the eye. Although the eye is illustrated in this example 600, it will be understood that the medical device can be attached to any biological tissue from any animal. The medical device can be attached to the surface of biological tissue, as illustrated in FIG. 6, or it can be implanted (e.g., attached to the sclera following a conjunctival incision and dissection, attached to an internal organ following surgical implantation, attached beneath the skin, attached to a vascular structure, attached to bone, attached to a lymph structure, attached to a nerve, including the spinal cord, attached to skin, attached to muscle, or the like). In addition, the device can be used in the treatment of systemic disease and can have utility for diagnostic studies and therapies (e.g., blood draws, IV placement, central line placement, lumbar punctures, inter-osseous drug treatments, catheter insertions, dialysis procedures, and the like).

An example of the medical device attached to an eye is shown here because the eye is an organ system that demonstrates the efficacy of the medical device in improving treatment modalities. For example, the medical device described herein can reduce both systemic toxicity and local toxicity inherent with ocular treatment modalities.

Many vision threatening ocular diseases are located in the retina. Systemic therapy for these diseases is limited by significant toxicity with proportionally low therapeutic yield. Additionally, the blood-retina barrier complicates drug delivery by preventing a number of therapeutic agents from reaching the retina. Due to the limitations of systemic drug delivery, significant effort has been placed into developing alternate methods that facilitate local drug delivery to the retina, including subconjunctival or transcleral delivery. Transcleral drug delivery operates on the principal that molecules of various sizes can diffuse through the sclera, reaching the choroid, vitreous, and the retina; however, depending on the nature of the agent injected and the number of subconjunctival or subtenons injections, local toxicity can be severe.

Intravitreal delivery has a number of significant advantages compared to transcleral delivery, including the rapid delivery of agents directly into the vitreous, and thus to the retina and choroid, which allows therapeutic drug levels to be achieved rapidly. It is limited by the fact that intravitreal drug levels peak with injection and then are rapidly reduced; however, the development of sustained release formulations of drugs, including nano-particle technology, is predicted to help deal with this limitation.

However, intravitreal injections, and any other procedure that penetrates into the eye, create a temporary communication between the outside and the inside of the eye. The communication is often signaled by the presence of vitreous reflux following removal of an injection device. This temporary channel greatly increases the risk of significant toxicity, including serious infection (endophthalmitis), and in the case of tumors contained within the eye, extraocular tumor spread (for example, retinoblastoma).

A key factor for increasing the safety of intravitreal injections and similar penetrating procedures is to address the temporary conduit formed following the penetration procedure. The medical device described herein is designed to address the temporary communication created by penetrating procedures like intravitreal injections. The medical device described herein can increase the safety of intravitreal injections by nature of its sealing property that allows the holding of an agent 204 under pressure. Following removal of a penetrating instrument 404, the agent 204 is forced into the temporary channel, treating this important site of potential toxicity.

According to one example, the medical device described herein can facilitate intravitreal injection. The medical device can have a shape on at least a portion of the medical device that can closely mirror the shape of the biological tissue. The injection port 404 can have markings to delineate the distance from the biological tissue (e.g., the limbus), thereby assisting in placement of the intravitreal injection. The medical device can be pre-filled with the agent at a predetermined pressure or be filled, re-filled, or emptied following attachment to the tissue. The medical device can be placed at any location on the eye, including being placed on the conjunctiva in the vicinity of the pars plana. The medical device can have dimensions that facilitate a certain penetrating device to penetrate through the device and into the eye tissue to a predetermined depth depending on the procedure.

In the case of endophthalmitis prophylaxis, the device can contain an antiseptic, such as povidone-iodine or betadine. Following attachment and sealing to the conjunctiva, the agent will be able to come in contact with the conjunctiva, sclera, or other ocular tissue at the release port, killing any infectious cells on that surface. When the needle penetrates the reservoir, any infectious cells on the needle will similarly be exposed to the antiseptic, thus reducing the chance of introducing infectious organisms into the eye. Following removal of the needle through the eye and device, the antiseptic in the device would flow into the temporary channel (needle track), killing any infectious cells in the track.

In the case of retinoblastoma, tumor cells inside the eye have been known to populate the needle track following intravitreal penetration procedures. The simple, sealable pressurizable medical device described herein can counteract this reflux by containing an agent toxic to the tumor cells. Following injections given through the medical device, this agent will be forced into the needle track, killing any cells that may be lodged in the needle track. In addition to increasing the safety profile of these injections, the agent contained in the device can potentiate the treatment.

The device can also be used, for example, in the treatment of organ-confined malignancy (e.g., renal tumor, pancreatic tumor, hepatic tumor, bladder tumor, breast tumor, prostate tumor, adrenal tumor, bone tumor, lung tumor, brain tumor, and the like). Another use can be augmenting the treatment of infectious cysts, abscesses, localized infections, including osteomyelitis, located in any part of the body, including those confined to a single organ system or multiple organ systems. It can be used in the diagnosis or treatment, medical or surgical, of any ocular disease.

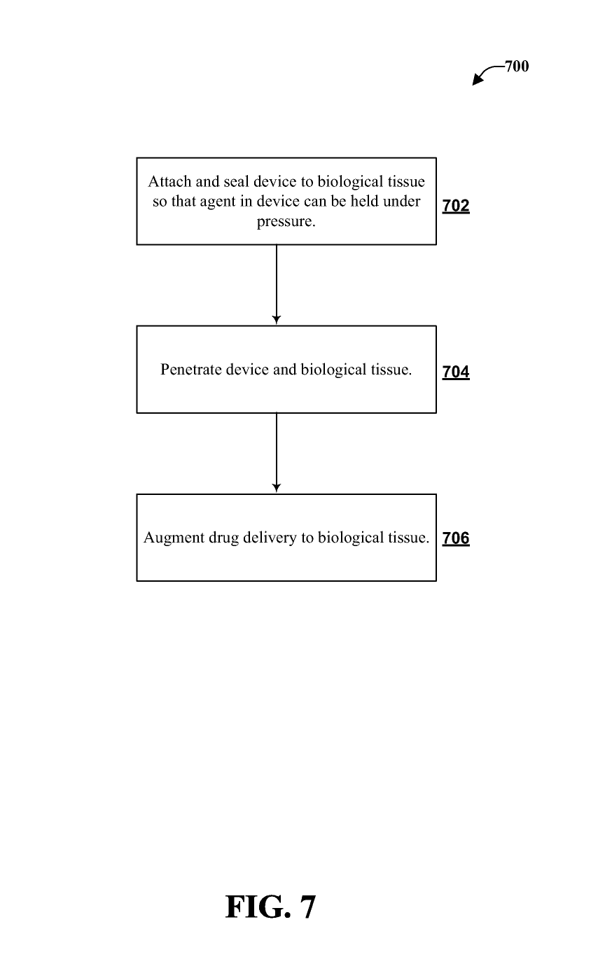

Referring now to FIGS. 7-9, illustrated are methods for delivering an agent to a biological tissue. For simplicity of explanation, anything described herein as a “method” is depicted and described as a series of acts. A system, apparatus or device can execute these methods to perform the acts.

It is to be understood and appreciated that the various embodiments are not limited by the acts illustrated and/or by the order of acts. For example, acts can occur in various orders and/or concurrently, and with other acts not presented or described herein. Furthermore, all illustrated acts may not be required to implement methods as described herein.

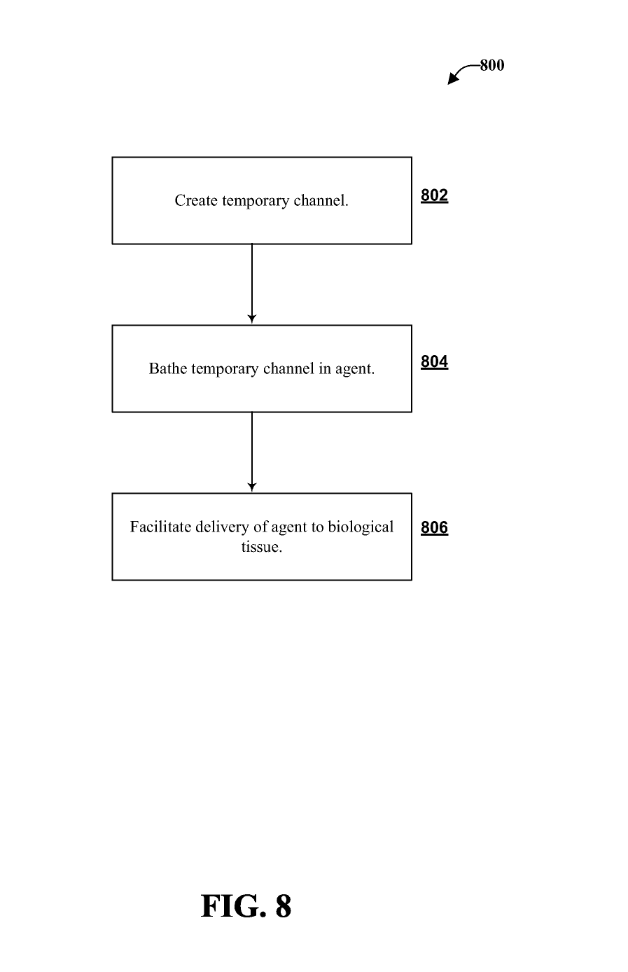

Referring now to FIG. 7, illustrated is a schematic process flow diagram of a method 700 for delivering an agent to biological tissue. At element 702, a device is attached to biological tissue. The device can house an agent under pressure. The attachment can be a fluid tight seal that can facilitate the agent being held under pressure in the device. At 704, the device and the underlying biological tissue can be penetrated by a penetrating device. At 706, drug delivery to the biological tissue can be augmented as shown in FIG. 8.

Referring now to FIG. 8, illustrated is a schematic process flow diagram of a method 800 for augmenting delivery of an agent to biological tissue. At element 802, a temporary channel can be created when the penetrating device is removed from the tissue. At element 804, the agent within the medical device is drawn or forced into the temporary channel to bathe the temporary channel in the agent. At element 806, delivery of the agent to the biological tissue can be facilitated.

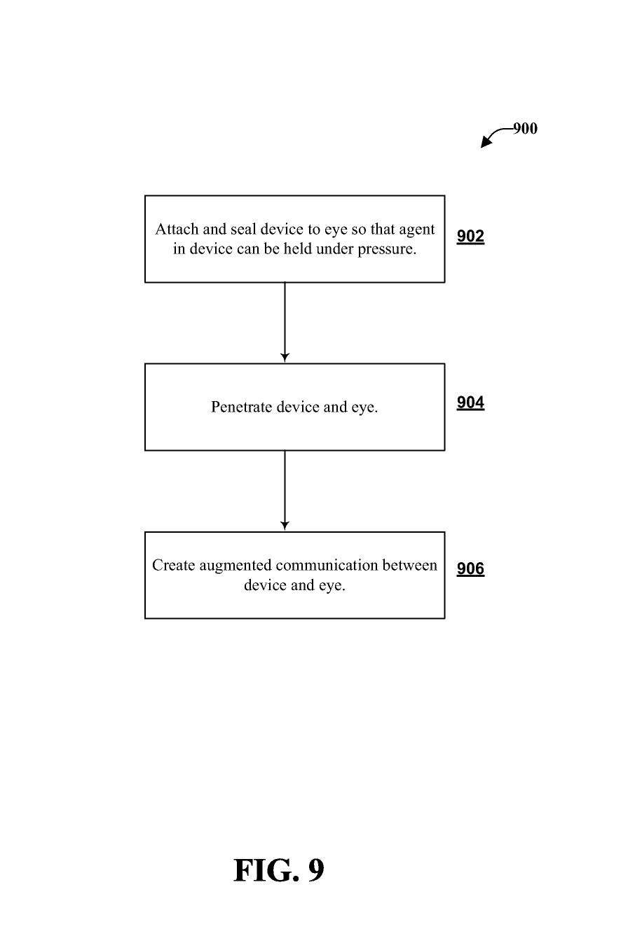

Referring now to FIG. 9, illustrated is a schematic process flow diagram of an example method 900 for delivery of an agent to a biological tissue: an eye. At element 902, a medical device holding an agent under pressure is attached to any part of the eye through any means. Alternatively, the device and penetrating instrument can be attached as a single system prior to attachment of the device to any part of the eye. At element 904, the medical device and the eye can be penetrated by a penetrating device. At element 906, communication between the device and the eye can be augmented, for example, by creating a temporary channel when the penetrating device is removed from the eye, bathing the temporary channel in an agent.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding Figures, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims.

What is claimed is:

1. A medical device, comprising:
a reservoir configured to hold an agent, the reservoir comprising a release port that facilitates delivery of the agent to a biological tissue and an impermeable portion that is impermeable to release of the agent; and
a sealable base connected to the reservoir, the sealable base comprising an attachment mechanism that facilitates an attachment to the biological tissue.

2. The medical device of claim 1, wherein the attachment mechanism is capable of rapid attachment to the biological tissue and removal from the biological tissue.

3. The medical device of claim 1, wherein the reservoir holds the agent prior to the attachment to the biological tissue.

4. The medical device of claim 1, wherein the agent has a medical application.

5. The medical device of claim 1, wherein the release port comprises a structural element to retain the agent in the reservoir prior to the attachment to the biological tissue.

6. The medical device of claim 1, wherein the impermeable portion comprises an injection port that facilitates filling, refilling, or emptying the reservoir while the medical device is attached to the biological tissue.

7. The medical device of claim 7, wherein the injection port is covered by a material that allows penetration with a penetrating instrument.

8. The medical device of claim 8, wherein the injection port is self-sealing upon removal of the penetrating instrument.

9. The medical device of claim 1, wherein the reservoir or the injection port comprises markings that facilitate administration of the agent to the reservoir or to the biological tissue.

10. The medical device of claim 1, wherein a size of the reservoir is variable based on an amount of the agent held in the reservoir with a corresponding change of pressure inside the reservoir based on the amount of the agent held in the reservoir.

11. The medical device of claim 1, wherein the release port is configured to open to biological tissue to facilitate release of the agent to the biological tissue.

12. The medical device of claim 1, wherein delivery of the agent to the biological tissue is augmented following penetration and removal of an instrument through the medical device and the biological tissue.

13. The medical device of claim 1, wherein the agent is used prophylactically to improve a safety profile of procedures that penetrate through the medical device and penetrate the biological tissue.

14. The medical device of claim 1, wherein the seal is a fluid tight seal.

15. A method, comprising:
attaching a sealable base of a device to a biological tissue, wherein the device comprises a reservoir containing an agent under pressure, the sealable base, an injection port, and a release port;
penetrating the device through the injection port, the reservoir and the release port with a penetrating instrument;
penetrating the biological tissue with the penetrating instrument; and
removing the penetrating instrument from the biological tissue and the medical device, augmenting delivery of the agent to the biological tissue.

16. A medical device, comprising:
a reservoir configured to hold an agent, wherein the reservoir comprises a release port that facilitates delivery of the agent to biological tissue and an impermeable portion that is impermeable to diffusion of the agent;
an injection port connected to the reservoir;
a sealable base connected to the reservoir, comprising an attachment mechanism that facilitates a fluid tight seal with the biological tissue; and
a pressurized system that facilitates the delivery of the agent to the biological tissue via a temporary channel created by penetration and subsequent removal of a penetrating device.

17. The medical device of claim 19, wherein the medical device is implantable.

18. The medical device of claim 19, wherein the penetrating device is a needle.

19. The medical device of claim 19, wherein the temporary channel is bathed in the agent from the reservoir.

20. The medical device of claim 19, wherein the sealable base comprises an air filled tube.

21. The medical device of claim 19, further comprising a holding portion mechanically coupled to the reservoir or the sealable base that facilitates stability and sealing related to the attachment to the biological tissue.
